(19) United States
(12) Patent Application Publication
(10) Pub. No.: US 2022/0151825 A1
(43) Pub. Date: May 19, 2022

(54) Self-Adhesive Cold Pack

(71) Applicant: Gary Leonard Mullen, Lincoln, DE (US)

(72) Inventor: Gary Leonard Mullen, Lincoln, DE (US)

(21) Appl. No.: 17/098,362

(22) Filed: Nov. 14, 2020

(51) Int. Cl. A61F 7/10

(52) U.S. Cl. CPC A61F 7/106

(57) ABSTRACT

The Self-Adhesive Cold Pack has a sealed impervious pocket also known as the reaction chamber that contains a cooling agent or material, that when mixed cause an endothermic reaction to occur. Affixing tabs are attached lengthwise to the top and bottom edges of the pocket. The affixing tabs are designed to adhere the cold pack to the skin of the patient or person during treatment.

### FIELD OF INVENTION

The present invention relates to the field of cold packs and medical for the application to an injury site on a body or a patient or person.

### BACKGROUND OF THE INVENTION

Instant cold packs are widely used in health care industries and private life to quickly aid in the treatment of injury. They can be stored at room temperature, eliminating the need for frozen storage. The instant cold pack can maintain its temperature for about 20-30 minutes. The cold packs are applied to the area of injury and held in place by hand for the duration of treatment. This process hinders the patient or person applying treatment from being productive either in the workplace or private life or administering other treatment that may be needed for the patient or individual being treated.

### BRIEF SUMMARY OF INVENTION

The invention is focused on a Self-Adhesive Cold Pack. While some instant cold packs eliminate the need for cold storage, the Self-Adhesive Cold Pack of the present invention eliminates the need to be held in place by hand.

### BRIEF DESCRIPTION OF DRAWINGS

FIG. 1 is a front view (side of the Self-Adhesive Cold Pack away from skin) of the Self-Adhesive Cold Pack 1.

FIG. 2 is a side view of the Self-Adhesive Cold Pack 1.

FIG. 3 is a back view (side of the Self-Adhesive Cold Pack applied to skin) of the Self-Adhesive Cold Pack 1 showing the top Affixing skin adhesion tab 3 with the skin adhesion protective cover releasing film paper 6 slightly pulled back exposing a portion of the skin adhesive material 5.

FIG. 4 is an exploded back view of the Self-Adhesive Cold Pack 1 showing the top affixing skin adhesion tab 3 with the skin adhesion protective cover releasing film paper 6 slightly pulled back exposing a portion of the skin adhesive material 5 and the bottom affixing skin adhesion tab 3 without the skin adhesion protective cover releasing film paper 6, exposing the entire skin adhesive material 5. In the center of the Self-Adhesive Cold Pack 1, FIG. 4 also shows the exploded view of the impervious pocket also known as the reaction chamber 2 with a cooling agent or material inside.

FIG. 5 is an exploded side view of the Self-Adhesive Cold Pack 1 showing the impervious pocket also known as the reaction chamber 2 containing the said same cooling agent or material as FIG. 4.

FIG. 6 is an exploded back view of the Self-Adhesive Cold Pack 101 showing the top affixing skin adhesion tab 103 with the skin adhesion protective cover releasing film paper 106 slightly pulled back exposing a portion of the skin adhesive material 105 and the bottom affixing skin adhesion tab 103 without the skin adhesion protective cover releasing film paper 106, exposing the entire skin adhesive material 105. In the center of the Self-Adhesive Cold Pack 101, FIG. 6 also shows the exploded view of the impervious pocket also known as the reaction chamber 102 containing an alternative cooling agent or material 110.

FIG. 7 is an exploded side view of the Self-Adhesive Cold Pack 101 showing the impervious pocket also known as the reaction chamber 102 containing the same said alternative cooling agent or material as FIG. 6.

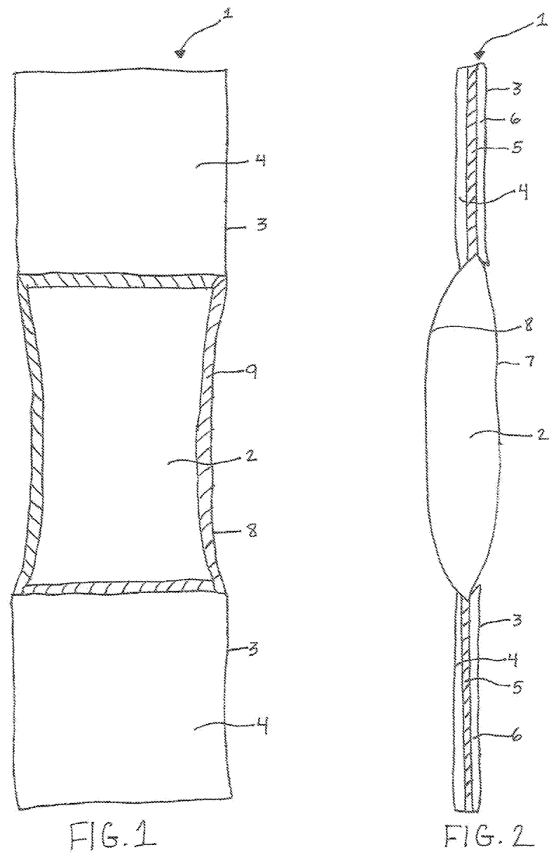

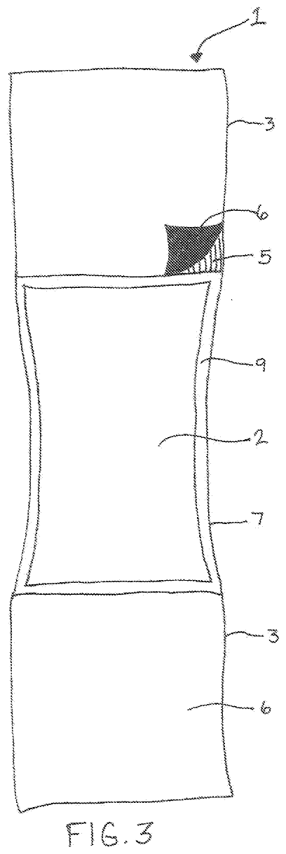

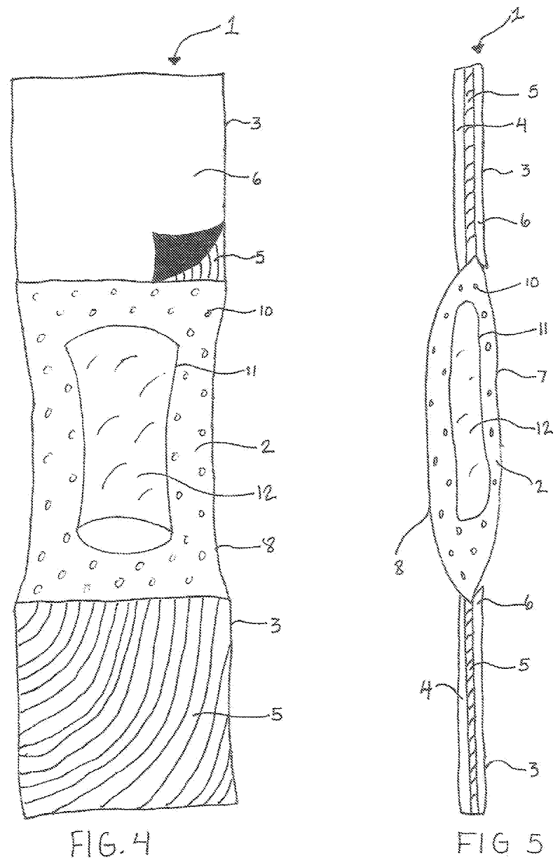

### DETAILED DESCRIPTION OF THE INVENTION

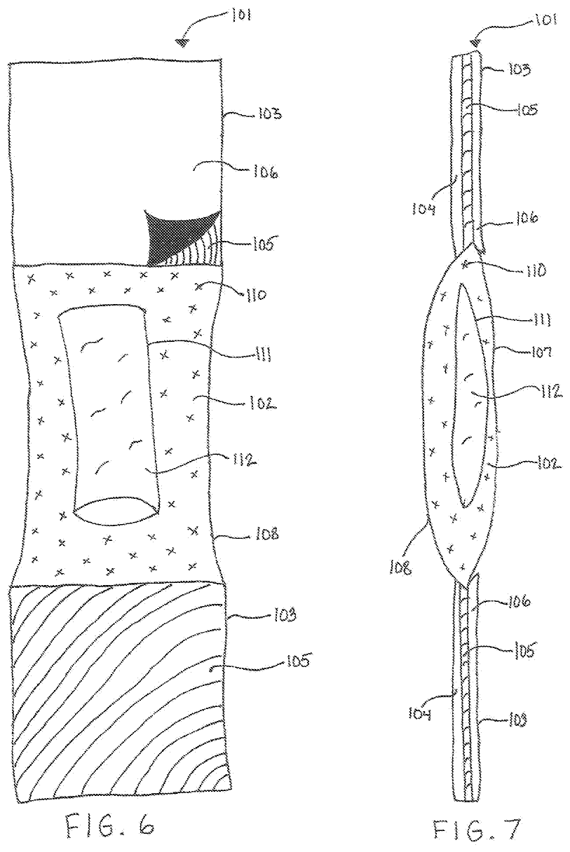

An embodiment to the preferred of the Self-Adhesive Cold Pack of the present invention is here and in figures disclosed. For clarity, within this document all reference to the front of the Self-Adhesive Cold Pack will correspond to the Self-Adhesive Cold Pack as viewed in FIG. 1 (the side of the Self-Adhesive Cold Pack away from the skin). All reference to the back of the Self-Adhesive Cold Pack will correspond to the Self-Adhesive Cold Pack as viewed in FIG. 3, FIG. 4, and FIG. 6.

The Self-Adhesive Cold Pack 1 is made of an impervious material that has a sealed pocket containing Ammonium Nitrate 10 and water 11, the cooling agent and material for cooling. The Self-Adhesive Cold Pack 1 is comprised of a front sheet 8 and a back sheet 7 of impervious material of which are equally cut sheets that are sealed 9 together on the right and left sides. An affixing skin adhesion tab 3 is then attached to the bottom end of the front impervious sheet 8 with the skin adhesion material 5 and the skin adhesion protective cover releasing film paper 6 facing downward and the polyurethane film 4 facing upward. The affixing skin adhesion tab 3 and the two impervious sheets 7 and 8 are then sealed 9 together on the bottom end of the impervious pocket 2. The Ammonium Nitrate 10 is then placed in the pocket 2 along with a rupturable bladder filled with water 11. An affixing skin adhesion tab 3 is then attached to the top end of the front impervious sheet 8. The affixing skin adhesion tab 3 and the two impervious sheets 7 and 8 are then sealed together on the top end of the impervious pocket 2 also known as the reaction chamber. When the Ammonium Nitrate 10 and water 11 are mixed, an endothermic reaction occurs

The second embodiment of the invention, The Self-Adhesive Cold Pack 101 is made of an impervious material that has a sealed pocket containing Ammonium Chloride 110 and water 111, the cooling agent and material for cooling. The Self-Adhesive Cold Pack 101 is comprised of a front sheet 108 and a back sheet 107 of impervious material of which are equally cut sheets that are sealed 109 together on the right and left sides. An affixing skin adhesion tab 103 is then attached to the bottom end of the front impervious sheet 108 with the skin adhesion material 105 and the skin adhesion protective cover releasing film paper 106 facing downward and the polyurethane film 104 facing upward. The affixing skin adhesion tab 103 and the two impervious sheets 107 and 108 are then sealed 109 together on the bottom end of the impervious pocket 102. The Ammonium Chloride 10 is then placed in the pocket 102 along with a rupturable bladder filled with water 111. An affixing skin adhesion tab 103 is then attached to the top end of the front impervious sheet 108. The affixing skin adhesion tab 103 and the two impervious sheets 107 and 108 are then sealed together on the top end of the impervious pocket 102 also known as the reaction chamber. When the Ammonium Chloride 110 and water 111 are mixed, an endothermic reaction occurs.

While preferred embodiments of the present invention, the Self-Adhesive Cold Pack, have been shown and described herein, the same is to be considered as illustrative and not restrictive in character, it being understood that only the preferred embodiments have been shown and described and that all changes and modifications that come within the spirit of the inventions are desired to be protected and covered thereby.

I claim:

1. A Self-Adhesive Cold Pack comprising:
a. An impervious flexible pocket also known as a sealed reaction chamber containing a cooling agent or material in said cold pack volume that can be stored at room temperature.
b. A flexible affixing tab attached to said pocket lengthwise from said top edge and said bottom edge of said pocket.
c. An adhesive means or method for attaching said affixing tabs to said pocket.
d. Adhesive means to temporarily adhere said cold pack to the skin.

2. The Self-Adhesive Cold Pack of claim 1 is comprised of a rupturable bladder within the said impervious pocket also know as the said reaction chamber.

3. The Self-Adhesive Cold Pack of said claim 1 wherein said cooling agent is made up of a first and second component. An endothermic reaction occurs when said first component and said second components are mixed.

4. The Self-Adhesive Cold Pack of claim 3 wherein said first component is water and said second component is ammonium nitrate.

5. The Self-Adhesive Cold Pack of claim 3 wherein said first component is water and an alternative said second component is ammonium chloride.

6. The Self-Adhesive Cold Pack of claim 1 where in said pocket could have multiple dimensions enabling the ability to adhere to any individual ranging from infants to adults and on any location of the body, from smaller areas to larger areas, but not limited to one size.
